(19) United States
(12) Patent Application Publication
(10) Pub. No.: US 2002/0025974 A1
(43) Pub. Date: Feb. 28, 2002

(54) BICYCLE THIOPHENES AND THIAZOLES

(76) Inventors: ELLEN W. BAXTER, GLENSIDE, PA (US); ROBERT E. BOYD, HORSHAM, PA (US); JOHN R. CARSON, NORRISTOWN, PA (US); MICHELLE C. JETTER, NORRISTOWN, PA (US); ALLEN B. REITZ, LANSDALE, PA (US)

Correspondence Address: AUDLEY A CIAMPORCERO JR, ONE JOHNSON AND JOHNSON PLAZA, NEW BRUNSWICK, NJ, 089337003

(21) Appl. No.: 09/442,175

(22) Filed: Nov. 16, 1999

Related U.S. Application Data
(60) Provisional application No. 60/108,935, filed on Nov. 18, 1998.

(51) Int. Cl. A61K 31/4178; A61K 31/427; C07D 277/02; C07D 233/00

(52) U.S. Cl. 514/367; 514/397; 548/152; 548/311.4

(57) ABSTRACT

Certain imidazo benzthiophenes/thiazoles of the formula:

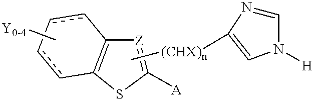

where

Z is N or C-Y (Y is absent where the imidazole containing substituent is attached at the 3-position);

n is 0 or 1;

Y is independently selected from the group consisting of hydrogen, C₁₋₄alkyl, bromine, chlorine, iodide, trifluoromethyl, C₁₋₄alkoxy, —SO₂NH₂ and nitro;

X is independently selected from the group consisting of hydrogen, hydroxy, C₁₋₄ alkyl, C₁₋₄ alkoxy, trifluoromethyl and phenyl; and

A is independently selected from the group consisting of hydrogen, C₁₋₄alkyl, chlorine, trifluoromethyl, C₁₋₄alkoxy and nitro, or is absent where the imidazole containing substituent is attached at the 2-position;

are α₂-adrenoceptor modulators which are useful in the treatment of hypertension, glaucoma, sexual dysfunction, depression, attention deficit hyperactivity disorder, the need for anesthesia, cardiac arrythmia or the need for analgesia.

The present invention relates to compounds which bind to the α₂-adrenoceptor. More particularly, the present invention relates to certain imidazo benzothiophenes/thiazoles which are α2 receptor modulators.

### BACKGROUND OF THE INVENTION

α₂-Adrenoceptor modulators are useful to treat a variety of conditions, including, hypertension, glaucoma, sexual dysfunction, depression, attention deficit hyperactivity disorder, the need for anesthesia, cardiac arrythmia and the need for analgesia. Particularly, α₂-adrenoceptor agonists are well known analgesics. α2-adrenoceptor antagonists have potential as antidepressants in their own right or as adjunct therapies to traditional inhibitors of monoamine reuptake.

Clonidine is a centrally acting α₂-adrenoceptor agonist with wide clinical utility as an antihypertensive agent. Clonidine is believed to act by inhibiting the release of norepinephrine from sympathetic nerve terminals via a negative feedback mechanism involving α₂-adrenoceptors located on the presynaptic nerve terminal. This action is believed to occur in both the central (CNS) and peripheral (PNS) nervous systems. More recently, the role of α₂-adrenoceptor agonists as analgesic agents in humans and antinociceptive agents in animals has been demonstrated. Clonidine and other α2-adrenoceptor agonists have been shown to produce analgesia through a non-opiate mechanism and, thus, without opiate liability. However, other behavioral and physiological effects were also produced, including sedation and cardiovascular effects.

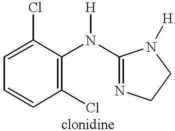

Medetomidine, detomidine, and dexmedetomidine are β₂-adrenoceptor agonists. Dexmedetomidine is used clinically in veterinary medicine as a sedatives/hypnotic for pre-anaesthesia. These compounds are hypotensive in animals and in humans, but the magnitude of this cardiovascular effect is relatively insignificant.

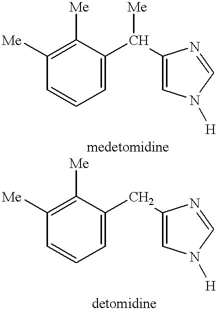

medetomidine detomidine

dexmedetomidine (dextrorotatory isomer)

U.S. Pat. No. 3,574,844, Gardocki et al., teach 4-[4(or 5)-imidazolylmethyl]-oxazoles as effective analgesics. The disclosed compounds are of the general formula:

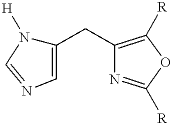

Compounds of this type are insufficiently active and suffer from unwanted side effects.

U.S. Pat. No. 4,913,207, Nagel et al., teach arylthiazolylimidazoles as effective analgesics. The disclosed compounds are of the general formula:

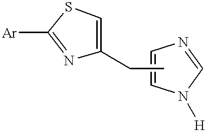

Compounds of this type are insufficiently active and suffer from unwanted side effects.

W092/14453, Campbell et al., teach 4-[(aryl or heteroaryl)methyl]-imidazoles as effective analgesics. The disclosed compounds are of the general formula:

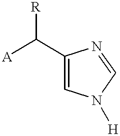

R is H or alkyl

A is aryl or heteroaryl

The disclosed compounds are insufficiently active and suffer from unwanted side effects.

Kokai No. 1-242571, Kihara et al., disclose a method to produce imidazole derivatives for use, among other uses, as antihypertensive agents.

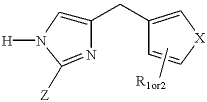

Z is H or phenyl

R is H, alkyl or halogen

X is S or O

A single mixture of compounds meeting the above formula was reportedly produced by the inventive method. This was a mixture of 4-(2-thienyl)-methylimidazole and 4-(3-thienyl)-methylimidazole represented by the following formula:

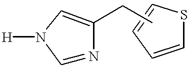

The disclosed compounds are insufficiently active and suffer from unwanted side effects.

U.S. Pat No. 5,621,113 and U.S. Pat No. 5,750,720, Boyd and Rasmussen disclose certain substituted 2- and 3-thienyl methylimidazoles as effective analgesic agents.

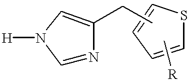

Many potent and selective α₂ antagonists, such as idazoxan, have been synthesized and evaluated in limited clinical trials as antidepressants.(J. Med. Chem. 1995, 38 (23), 4615.) Mirtazapine is a closely related analog of the established antidepressant mianserin. This compound has been shown to be an antagonist at α₂ receptors and exhibits antidepressant activity in vivo. (Exp. Opin. Invest. Drugs 1995, 4(10), 945). An agent with the dual profile of a 5 HT reuptake inhibitor and an α₂ antagonist might serve to enhance synaptic concentrations of 5-HT relative to that achievable through 5-HT uptake inhibition alone and in turn produce a more effective antidepressant response. A novel series of compounds with such a profile was found to possess putative antidepressant effects in vivo (J. Med. Chem. 1997, 40 (7), 1049; Bioorg. Med. Chem Lett. 1995, 5, 2287.; Drug. Dev. Res. 1995, 35, 237.; Drug. Dev. Res. 1995, 35, 246.)

### SUMMARY OF THE INVENTION

Briefly, there is provided by the present invention compounds which are α₂-adrenoceptor modulators of the formula:

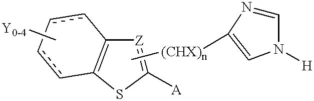

where

Z is N or C-Y (Y is absent where the imidazole containing substituent is attached at the 3-position);

n is 0 or 1;

Y is independently selected from the group consisting of hydrogen, C₁₋₄alkyl, bromine, chlorine, iodide, trifluoromethyl, C₁₋₄alkoxy, —SO₂NH₂ and nitro;

X is independently selected from the group consisting of hydrogen, hydroxy, C₁₋₄ alkyl, C₁₋₄ alkoxy, trifluoromethyl and phenyl; and

A is independently selected from the group consisting of hydrogen, C₁₋₄alkyl, chlorine, trifluoromethyl, C₁₋₄alkoxy and nitro, or is absent where the imidazole containing substituent is attached at the 2-position .

### DETAILED DESCRIPTION OF THE INVENTION

The compounds of the present invention are prepared by the methods shown in Scheme 1-4. In Scheme 1, an appropriately substituted benzthiophene aldehyde 1 is reacted with the Grignard reagent derived from N¹-trityl-4-iodoimidazole and ethyl or methylmagnesiumbromide (Turner, Lindell and Ley, J. Org. Chem., 1991, 5739). The resultant alcohol 2 can be deprotected under acidic conditions such as acidic methanol or trifluoroacetic acid in dichloromethane to give target 3. This same alcohol 2 could also be deoxygenated and deprotected in one step by reaction with triethylsilane and trifluoroacetic acid to give product 4. Scheme 1 is exemplified with a thiophen-2-yl, but can analogously be applied to produce thiophen-3-yl equivalents to compounds 2, 3 and 4 from an analogous thiophen-3-yl aldehyde 1.

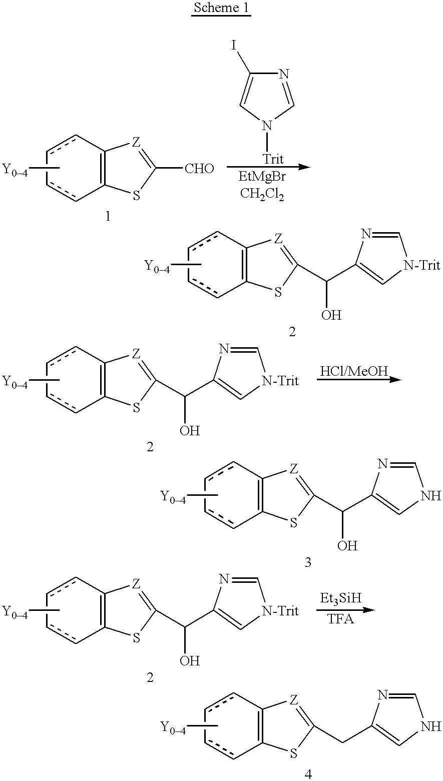

As depicted in Scheme 2, alcohol 2 could alternatively be obtained by the condensation of N¹-tritylimidazole-4-carboxaldehyde (Jetter, Boyd and Reitz, OPPI Briefs, 1996, 709) with the anion generated by reaction of the appropriately substituted thiophene with a strong base such as n-butyllithium. Scheme 2 is exemplified to produce thiophen-2-yl. However, the scheme may be modified to produce thiophen-3-yl where the benzothiophene starting material has a blocking substituent in the 2-position selected from the group, trialkylsilyl, C₁₋₄alkyl, chlorine, trifluoromethyl, C₁₋₄alkoxy and nitro. The trialkylsilyl group may be removed from analogous compound 2 by acid treatment or treatment with fluoride ion to leave a hydrogen substituent.

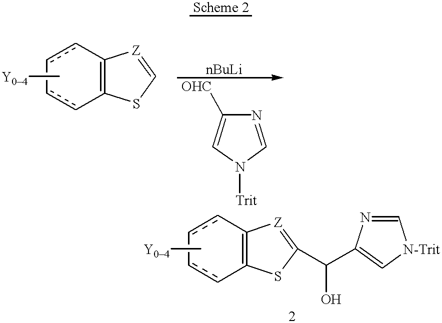

In Scheme 3, alcohol 2 is oxidized with a suitable oxidizing agent such as MnO₂ or pyridinium chlorochromate (PCC) to yield the ketone 5. This ketone reacts with an alkyl or phenylmagnesium halide (or other Grignard or lithium reagent) to give 6 which is then deoxygenated and deprotected in one step by hydrogenation using Pd(OH)₂ as catalyst to yield 7. Other means of deoxygenation/deprotection include triethylsilane/trifluoroacetic acid, 57% hydriodic acid, borane/methylsulfide or NaBH₄/trifluoroacetic acid. . Scheme 3 is exemplified with a thiophen-2-yl, but can analogously be applied to produce thiophen-3-yl equivalents to compound 7 from an analogous thiophen-3-yl alcohol 2.

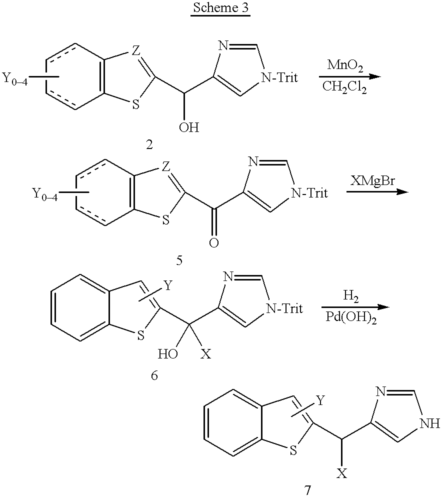

Scheme 4 illustrates the synthetic route to those compounds with no carbon chain between the thiophene analog and the imidazole. The appropriately substituted aldehyde 1 is reacted with tosylmethylisocyanide (TOSMIC) in the presence of a catalytic amount of sodium cyanide in ethanol to give the trans-4-tosyloxazolines 8. The intermediate isoxazolines 8 are heated in a saturated solution of ammonia in methanol to yield the imidazoles 9 as the final products. (Home, Yakushijin and Buchi, Heterocycles, 1994, 139.). Scheme 4 is exemplified with a thiophen-2-yl, but can analogously be applied to produce thiophen-3-yl equivalents to compound 9 from an analogous thiophen-3-yl aldehyde 1.

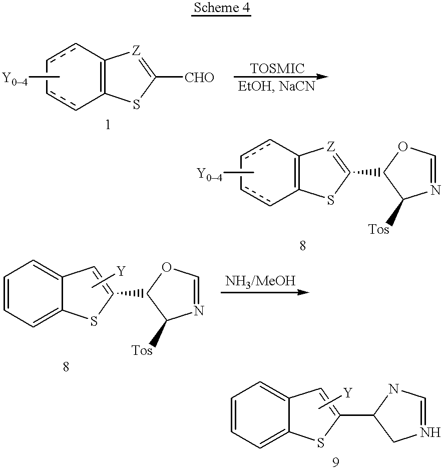

In Scheme 5, imidazole thioamide 10 is reacted with an appropriate cycloalkyl haloketone to give the fused thiazole 11. The necessary thioamide 10 is synthesized by reaction of the imidazole acetonitrile 12 as shown below with thioacetamide. Imidazole acetonitrile 12 was obtained from histidine by published procedures as disclosed by Hirsch and Richardson in J. Appl. Chem., 1969, 83.

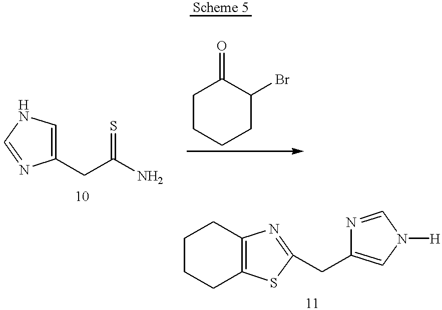

As depicted in Scheme 6, imidazole acetonitrile 12 is reacted with an appropriately substituted aminothiophenol in an appropriate solvent such as methanol or ethanol to give the benzthiazole methylimidazole 13.

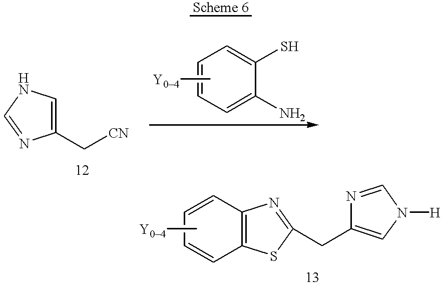

In the case where Y is hydrogen, C₁₋₄alkyl, C₁₋₄alkoxy and trifluoromethyl, the appropriately substituted thiophene hydroxyimidazole 2 may be produced and the substituent in question will stably endure the reactions of Scheme 1, Scheme 2 and Scheme 3 to arrive at target products 3, 4, 7 and 9. In the case where Y is chlorine, bromine, and nitro, the above described deoxygenation/deprotection steps could also be achieved using triethylsilane/trifluoroacetic acid, 57% hydriodic acid or other means such as borane/methylsulfide or NaBH₄/trifluoroacetic acid.

The compounds of the present invention may be used to treat a medical condition as named herein, such as, mild to moderate pain in warm-blooded animals, such as, humans by administration of an effective dose. The dosage range would be from about 10 to 3000 mg, in particular about 25 to 1000 mg or about 100 to 500 mg, of active ingredient 1 to 4 times per day for an average (70 kg) human although it is apparent that activity of individual compounds of the invention will vary as will the pain being treated. In regard to the use of these α₂-adrenoceptor modulators to treat hypertension, glaucoma, sexual dysfunction, depression, attention deficit hyperactivity disorder, the need for anesthesia and cardiac arrythmia, a therapeutically effective dose can be determined by persons skilled in the art by use of established animal models. Pharmaceutical compositions of the invention comprise the formula (I) compounds as defined above, particularly in admixture with a pharmaceutically-acceptable carrier.

To prepare the pharmaceutical compositions of this invention, one or more compounds of the invention or salt thereof as the active ingredient, is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above.

The pharmaceutically acceptable salts referred to above generally take a form in which the imidazolyl ring is protonated with an inorganic or organic acid. Representative organic or inorganic acids include hydrochloric, hydrobromic, hydroiodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benezenesulfonic, oxalic, pamoic, 2-naphthalenesulfonic, p-toluenesulfonic, cyclohexanesulfamic, salicylic or saccharic.

Biological Procedures

The activity of compounds of the invention as α₂ modulators may be demonstrated by the in vivo and in vitro assays as described below:

Alpha-2D adrenergic receptor binding assay

Male, Wistar rats (150-250 g, VAF, Charles River, Kingston, N.Y.) are sacrificed by cervical dislocation and their brains removed and placed immediately in ice cold HEPES buffered sucrose. The cortex is dissected out and homogenized in 20 volumes of HEPES sucrose in a Teflon™-glass homogenizer. The homogenate is centrifuged at 1000 g for 10 min, and the resulting supernatant centrifuged at 42,000 g for 10 min. The resulting pellet is resuspended in 30 volumes of 3 mM potassium phosphate buffer, pH 7.5, preincubated at 25° C. for 30 min and recentrifuged. The resulting pellet is resuspended as described above and used for the receptor binding assay. Incubation is performed in test tubes containing phosphate buffer, 2.5 mM MgCl₂, aliquots of the synaptic membrane fraction, the ligand ³H-para-aminoclonidine and test drug at 25° C. for 20 min. The incubation is terminated by filtration of the tube contents through glass fiber filter sheets. Following washing of the sheets with 10 mM HEPES buffer, the adhering radioactivity is quantified by liquid scintillation spectrometry.

Binding of the test drug to the receptor is determined by comparing the amount of radiolabeled ligand bound in control tubes without drug to the amount of radiolabeled ligand bound in the presence of the drug. Dose-response data are analyzed with LIGAND, a nonlinear curve fitting program designed specifically for the analysis of ligand binding data. This assay is described by Simmons, R. M. A., and Jones, D. J., Binding of [³H-]prazosin and [³H-]p-aminoclonidine to α-Adrenoceptors in Rat Spinal Cord, Brain Research 445:338-349, 1988.

Mouse Acetylcholine Bromide-Induced Abdominal Constriction Assay (MAIT)

The mouse acetylcholine bromide-induced abdominal constriction assay, as described by Collier et al. in Brit. J. Pharmacol. Chem. Ther., 32: 295-310, 1968, with minor modifications was used to assess analgesic potency of the compounds herein. The test drugs or appropriate vehicle were administered orally (p.o.) and 30 minutes later the animal received an intraperitoneal (i.p.) injection of 5.5 mg/kg acetylcholine bromide (Matheson, Coleman and Bell, East Rutherford, N.J.). The mice were then placed in groups of three into glass bell jars and observed for a ten minute observation period for the occurrence of an abdominal constriction response (defined as a wave of constriction and elongation passing caudally along the abdominal wall, accompanied by a twisting of the trunk and followed by extension of the hind limbs). The percent inhibition of this response to a nociceptive stimulus (equated to % analgesia) was calculated as follows: The % Inhibition of response, i.e., % analgesia is equal to the difference between the number of control animals responding and the number of drug-treated animals responding times 100 divided by the number of control animals responding.

At least 15 animals were used for control and in each of the drug treated groups. At least three doses were used to determine each dose response curve and ED₅₀ (that dose which would produce 50% analgesia). The ED₅₀ values and their 95% fiducial limits were determined by a computer assisted probit analysis.

Biological Data

Table 1 lists compounds made in the examples below with certain biological data.

TABLE 1

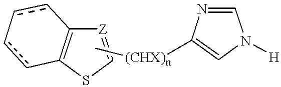

| | | | | Fused ring | | | |
|---|---|---|---|---|---|---|---|
| | 2 or 3 | | | sat or | | α_{2D} Kᵢ | |
| Cpd # | thienyl | X | Z | arom* | n | (nM) | MAIT |
| Cp-1 | Thien-2-yl | H | CH | Arom | 1 | 1.1 | 20 po |
| Cp-2 | Thien-2-yl | OH | CH | Arom | 1 | 74.9 | 27 po |
| Cp-3 | Thien-2-yl | Me | CH | Arom | 1 | 14 | 27 po |
| Cp-4 | Thien-2-yl | H | C—Br | Arom | 1 | 0.22 | 20 po |
| Cp-5 | Thien-2-yl | H | C—Me | Arom | 1 | — | 47 po |
| Cp-6 | Thien-3-yl | H | CH | Arom | 1 | 0.09 | 100 po |
| Cp-7 | Thien-2-yl | H | CH | Sat | 1 | 3.7 | 7 po |
| Cp-8 | Thien-2-yl | Me | CH | Sat | 1 | 40 | 20 po |
| Cp-9 | Thien-2-yl | — | CH | Mom | 0 | 18 | 40 po |
| Cp-10 | Thien-2-yl | — | C—Me | Arom | 0 | 0.031, | 14 po |
| | | | | | | 2.1 | |
| Cp-11 | — | H | N | Sat | 1 | 110 | 13 po |
| Cp-12 | — | H | N | Arom | 1 | 18 | 7 po |

*sat indicates fully saturated and arom indicates fully aromatic

The following Examples illustrate the invention:

### EXAMPLE 1

### Cp-1

Thianaphthene (0.04 moles) was dissolved in 100 ml of dry THF. The clear solution was cooled to −78° C. (dry ice/acetone) and stirred at −78° C. for 10 minutes. To this solution was added n-butyl lithium (0.044 moles) via syringe and the reaction mixture was stirred at −78° C. for 1 hour. N-formyl piperidine (0.04 mole) was then added and the reaction mixture was stirred at −78° C. for an additional 1.5 hours. The clear pale yellow reaction mixture was allowed to warm to room temperature and stirred for 1 hour at which time TLC (70:30 hex/EtOAc) indicated the disappearance of starting material. Saturated NH₄Cl (30 mL ) was added to the reaction mixture and it was extracted with CH₂Cl₂ (2×50 ml). The organic layer was dried over Na₂SO₄ and evaporated under reduced pressure. The residue was chromatographed on silica gel eluting with 70:30 hexane/EtOAc. The product was obtained as yellow oil (75% yield).

Ethyl magnesium bromide (8.0 mL, 3M in diethylether) was added slowly to a solution of 4-iodo-1-trityl-1H-imidazole in methylene chloride (100 mL) and stirred at room temperature. After 45 min a solution of thianaphthene-2-carboxaldehyde in methylene chloride (10 mL) was added slowly and stirred at room temperature forl6 hours. The reaction was quenched by addition of saturated NH₄Cl (20 mL) and washed sequentially with H₂O. The organic layer was dried over Na₂SO₄, filtered and the solvent evaporated under reduced pressure. The residue was chromatographed on silica gel eluting with 50:50 hexane/EtOAc to yield the product (65% yield) as a fluffy white solid. (Cp-2)

¹H NMR (CDCl₃):δ6 4.2 (bs, 1H, OH), 6.05 (s, 1H, CH), 6.8 (s, 1H, imidazole H-5), 7.0-7.3 (m, 16H, aromatic), 7.1 (s, 1H, thiophene), 7.5 (s, 1H, imidazole H-2), 7.6 (d, 1H, aromatic), 7.8 (d, 1H, aromatic)

Alternatively, Cp-2 could be synthesized by the condensation of N¹-tritylimidazole-4-carboxaldehyde with the thianaphthene anion. Thianaphthene (0.02 moles) was dissolved in 50 ml of dry THF. The clear solution was cooled to −78° C. (dry ice/acetone) and stirred at −78° C. for 10 minutes. To this solution was added n-butyl lithium (0.022 moles) via syringe and the reaction mixture was stirred at −78° C. for 1 hour. N¹-tritylimidazole-4-carboxaldehyde (0.02 moles) obtained by published procedures (Jetter, Boyd and Reitz, OPPI Briefs, 1996, 709) was added to the reaction mixture in one portion and the reaction mixture was stirred at −78 for 1 hour. The clear pale yellow reaction mixture was then allowed to warm to room temperature. TLC analysis (70:30 hex/EtOAc) indicated the disappearance of starting material. Saturated NH₄Cl (30 mL ) was added to the reaction mixture and the reaction mixture was extracted with CH₂Cl₂ (2×50 ml). The organic layer was dried over Na2SO₄ and evaporated under reduced pressure. The residue was chromatographed on silica gel eluting with 70:30 hexane/EtOAc. The product was obtained as yellow oil (75% yield).

The above product, Cp-2 (0.01 mole), was dissolved in CH₂Cl₂ (50 mL). Trifluoroacetic acid (32 equivalents) was slowly added and the reaction mixture turned clear dark yellow. Triethylsilane was then slowly added to the reaction mixture via syringe and the clear dark yellow-orange solution was stirred at room temperature for 16 hours. H₂O(20 ml was added to the reaction mixture and the aqueous layer was made basic by the addition of solid Na₂CO₃. The organic layer was separated, washed with brine (20 mL) and dried over K₂CO₃. The solvent was evaporated under reduced pressure and the residue was chromatographed on silica gel eluting with a gradient of 5 50:50 hexane/EtOAc to 10:90 hexane/EtOAc. The product was obtained as a clear oil. (Cp-1)

¹H NMR (CDCl₃):δ4.2 (s, 2H benzyl CH₂), 6.9 (s, 1H, imidazole H-5), 7.1 (s, 1H, thiophene), 7.2-7.3 (m, 3H, aromatic), 7.6 (s, 1H, imidazole H-2), 7.7 (d, 1H, aromatic), 7.8 (d, 1H, aromatic).

1.0 M HCl in diethylether was added to the oil to form the hydrochloride salt.

The product was obtained as a light yellow solid.

¹H NMR (CD₃OD):δ4.3 (s, 2H, benzyl CH₂), 7.1 (s, 1H, thiophene), 7.1 (s, 1H, imidazole H-5), 7.3-7.4 (m, 2H, aromatic), 7.65 (d, 1H, aromatic), 7.75 (d, 1H, aromatic), 8.2 (s, 1H, imidazole H-2).

### EXAMPLE 2

Cp-4, Cp-5, Cp-6 and Cp-7 were synthesized in a manner analogous to the synthesis of Cp-1.

### EXAMPLE 3

### Cp-8

The imidazole alcohol, Cp-2, (3.1 g, 6.5 mmol) and MnO₂ (6 g) were combined in 50 mL of CH₂Cl₂ and reaction was stirred at room temperature. After 4 hrs the reaction was filtered through dicalite and the solvent was evaporated in vacuo. The residue was triturated with Et₂O and filtered to yield the imidazole ketone product as a yellow solid.

To a solution of the above mentioned imidazole ketone (2.6 g, 5.5 mmol) in THF (30 mL) was added MeMgBr (3.0 M, 4.5 mL). After the starting material was consumed, the reaction was quenched with aqueous NH₄Cl and extracted with EtOAc (2×). The combined extracts were washed with water and then brine and dried over Na₂SO₄. The solution was filtered and the solvent was evaporated in vacuo. The residue was dissolved in EtOH and 1N HCl (6 mL). Pd(OH)₂ (1.5 g) was added and the mixture was hydrogenated at 50° C., 50 psi overnight. The catalyst was removed by filtration and the filtrate was evaporated in vacuo. The residue was chromatographed on silica (99/0.75/0.25 EtOAc/MeOH/NH₄OH) to give an oil. This was combined with 1 eq. of fumaric acid in 2-PrOH. The solvent was evaporated in vacuo and the residue was recrystallized from acetone to give the title compound, Cp-8 (0.23 g, 12%), mp 144-147° C. ¹H NMR (DMSO-d₆) 1.55 (d, 3H), 1.65 (m, 4H), 2.45 (m, 2H), 2.65 (m, 2H), 4.2 (q, 1H), 6.5 (s, 1H), 6.65 (s, 2H), 6.8 (s, 1H), 7.55 (s, 1H). Anal Calc for C₁₃H₁₆N₂S·C₄H₄O₄C, 58.61; H, 5.79; N, 8.04. Found C, 58.44; H, 5.90; n, 7.85

### EXAMPLE 4

Cp-3 was synthesized in a manner analogous to the synthesis of Cp-8.

### EXAMPLE 5

### Cp-10

3-Methyl-2-thiophene carboxaldehyde (0.015 mole) and TOSMIC (tosylmethyisocyanide, 0.015 mole) were suspended in 30 ml of ethanol. A catalytic amount of NaCN (0.0015 mole) was added and the reaction mixture was stirred at room temperature. Gradually, the reaction mixture became homogeneous and turned clear dark-brown in color. After 40 minutes of stirring at room temperature, the reaction mixture turned cloudy and a precipitate was evident. Stirring was continued for a total of 2 hours until the starting material was consumed as evidenced by TLC (7:3 hexane /ethyl acetate) The reaction mixture was filtered and the collected solid was washed with 20 ml of hexane.

The intermediate oxazoline was suspended in 20 ml of 2.0 M NH₃/MeOH in a pressure flask. The reaction mixture was heated at 90° C. (oil bath) for 20 hours. Within the first hour, the reaction mixture became homogeneous and turned dark red in color. After 20 hours, the reaction mixture (dark brown) was cooled to room temperature and the solvent was evaporated. The residue was taken up in ethyl acetate and extracted with 1.0 N HCl (30 ml). The aqueous layer was then basified with solid Na₂CO₃ and extracted with ethyl acetate (2×30 ml). The organic layer was washed with brine (25 ml), dried over K₂CO₃ and evaporated. The residue was chromatographed on silica gel eluting with CH₂Cl₂/ 5% MeOH to yield the product as a yellowish solid. (Cp-10)

¹H NMR (CD₃OD): d 2.50 (s, 3H, CH₃), 7.3-7.4 (m, 3H, aromatic, imidazole H-5), 7.7-7.8 (m, 3H, aromatic, imidazole H-2).

1.0 M HCl in diethylether was added to the solid to yield the HCl salt which was recrystallized from acetone to give the product as a pale yellow solid.

¹H NMR (CD₃OD): d 2.50 (s, 3H, CH₃), 7.4-7.45 (m, 2H, aromatic, imidazole H-5), 7.8-7.9 (m, 3H, aromatic), 8.9 (s, 1H, imidazole H-2).

### EXAMPLE 6

Cp-9 was synthesized in a manner analogous to Cp-10.

### EXAMPLE 7

### Cp-11

Imidazol-4-yl acetonitrile (12.8 g, 120 mmol) and thioacetamide (18.0 g, 240 mmol) were combined in 150 mL of dry DMF. Dry HCl gas was bubbled into the reaction mixture as it was heated in a 90-100° C. oil bath. The starting material was consumed in about 45 minutes as judged by TLC. The solvent was evaporated in vacuo and the residue was triturated with acetone. The crude product was collected by filtration and recrystallized from 2-propanol (2×) to give the imidazole thioacetamide as a beige solid (16.2 g, 76%), mp 199-201° C.

2-Bromocyclohexanone (3.0 g, 17 mmol) and (imidazol-4-yl) thioacetamide hydrochloride as synthesized above (3.0 g, 17 mmol) were combined in MeOH (10 mL) and heated at reflux. An additional 1.5 g of 2-bromocyclohexanone was added and reflux continued for 3 hrs. The solvent was evaporated in vacuo and the residue partitioned between water and Et₂O. The aqueous layer was washed with one additional portion of Et₂O, and then basified and extracted with CHCl₃ (2×). The extracts were combined, dried over K₂CO₃, filtered and the solvent was evaporated in vacuo. The crude product was dissolved in 2-propanol, treated with fumaric acid to a pH of 4 and allowed to stand at room temperature overnight. The product was collected by filtration and recrystallized from 2-propanol to give the title compound as an off-white solid, Cp-11 (0.7 g, 10%), mp 169-171° C. ¹H NMR (DMSO-d₆) 1.8 (m, 4H), 2.65 (m, 4H), 4.1 (s, 2H), 6.65 (s, 3H), 7.0 (s, 1H), 7.65 (s, 1H). Anal calc for C₁₁H₁₃N₃S.1.5 C₄H₄O₄ C, 51.90; H, 4.87; N, 10.68. Found C, 51.93; H, 4.83; N, 10.58.

### EXAMPLE 8

### Cp-12

A solution of imidazol-4-yl acetonitrile (3.0 g, 28 mmol) and 2-amino-benzenethiol hydrochloride (5.0 g, 30 mmol) in EtOH (75 mL) was heated at reflux overnight. The solvent was evaporated in vacuo and the residue was dissolved in 3N HCl and washed with Et₂O (2×). The aqueous layer was basified with Na₂CO₃ and extracted with EtOAc (2×). The organic extracts were combined and dried over K₂CO₃, filtered and the solvent was evaporated in vacuo. The residue was dissolved in acetone and treated with 1 eq. of fumaric acid which was dissolved in a minimum amount of 2-propanol. The solid was collected by filtration and recrystallized from acetone to give the title compound as an off-white solid, Cp-12 (1.2 g, 11%), mp 186-187° C. ¹H NMR (DMSO-d₆) 4.4 (s, 2H), 6.65 (s, 3H), 7.1 (s, 1H), 7.45 (2t, 2H), 7.7 (s, 1H), 8.0 (2d, 2H). Anal Calc for C₁₁H₉N₃S.1.5C₄H₄O₄ C, 52.44; H, 3.88; N, 10.79. Found C, 52.53; H, 3.98; N, 10.74.

Table 2 summarizes the mass spec data for the compounds of Table 1.

TABLE 2

| Compound # | Mass (calc) | Mass (obs) |
|---|---|---|
| Cp-1 | 214.3 | 215 |
| Cp-2 | 230.3 | 231 |
| Cp-3 | 228.3 | 229 |
| Cp-4 | 293.2 | 294 |
| Cp-5 | 228.3 | 229 |
| Cp-6 | 214.3 | 215 |
| Cp-7 | 218.3 | 219 |
| Cp-8 | 232.4 | 233 |
| Cp-9 | 200.3 | 201 |
| Cp-10 | 214.3 | 215 |
| Cp-11 | 219.3 | 220 |
| Cp-12 | 215.3 | 215 |

What is claimed is:

1. The α₂-adrenoceptor modulators of the formula: 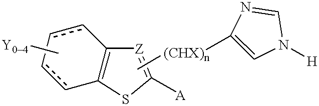
where
Z is N or C-Y (Y is absent where the imidazole containing substituent is attached at the 3-position);
n is 0 or 1;
Y is independently selected from the group consisting of hydrogen, C₁₋₄alkyl, bromine, chlorine, iodide, trifluoromethyl, C₁₋₄alkoxy, -SO₂NH₂ and nitro;
X is independently selected from the group consisting of hydrogen, hydroxy, C₁₋₄ alkyl, C₁₋₄ alkoxy, trifluoromethyl and phenyl; and
A is independently selected from the group consisting of hydrogen, C₁₋₄alkyl, chlorine, trifluoromethyl, C₁₋₄alkoxy and nitro, or is absent where the imidazole containing substituent is attached at the 2-position.

2. The compound of claim 1 which is a α₂-adrenoceptor agonist.

3. The compound of claim 1 which is a α₂-adrenoceptor antagonist.

4. The compound of claim 1 which has the general formula: 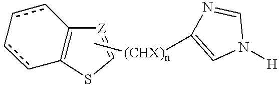
wherein the position of thienyl substitution, X, Z, saturation pattern and n are dependently selected from the groups consisting of:

| | | | | Fused ring | |
|---|---|---|---|---|---|
| Cpd # | 2 or 3 thienyl | X | Z | sat or arom | n |
| Cp-1 | Thien-2-yl | H | CH | Arom | 1 |
| Cp-2 | Thien-2-yl | OH | CH | Arom | 1 |
| Cp-3 | Thien-2-yl | Me | CH | Arom | 1 |
| Cp-4 | Thien-2-yl | H | C-Br | Arom | 1 |
| Cp-5 | Thien-2-yl | H | C-Me | Arom | 1 |
| Cp-6 | Thien-3-yl | H | CH | Arom | 1 |
| Cp-7 | Thien-2-yl | H | CH | sat | 1 |
| Cp-8 | Thien-2-yl | Me | CH | sat | 1 |
| Cp-9 | Thien-2-yl | — | CH | Arom | 0 |
| Cp-10 | Thien-2-yl | — | C-Me | Arom | 0 |
| Cp-11 | — | H | N | sat | 1 |
| Cp-12 | — | H | N | Arom | 1 |

5. A method for treating a human suffering from hypertension, glaucoma, sexual dysfunction, depression, attention deficit hyperactivity disorder, the need for anesthesia, cardiac arrythmia or the need for analgesia comprising the step of administering an effective amount of a compound which is a suitable α₂-adrenoceptor modulator of the formula: 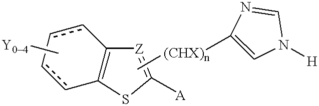
where
Z is N or C-Y (Y is absent where the imidazole containing substituent is attached at the 3-position);
n is 0 or 1;
Y is independently selected from the group consisting of hydrogen, C₁₋₄alkyl, bromine, chlorine, iodide, trifluoromethyl, C₁₋₄alkoxy, —SO₂NH₂ and nitro;
X is independently selected from the group consisting of hydrogen, hydroxy, C₁₋₄ alkyl, C₁₋₄ alkoxy, trifluoromethyl and phenyl; and
A is independently selected from the group consisting of hydrogen, C₁₋₄alkyl, chlorine, trifluoromethyl, C₁₋₄alkoxy and nitro, or is absent where the imidazole containing substituent is attached at the 2-position .

6. The method of claim 5 wherein said human is suffering from the need for analgesia and said compound is a α₂-adrenoceptor agonist.

7. The method of claim 5 wherein said human is suffering from depression and said compound is a α₂-adrenoceptor antagonist.

8. A pharmaceutical preparation for treating a human suffering from hypertension, glaucoma, sexual dysfunction, depression, attention deficit hyperactivity disorder, the need for anesthesia, cardiac arrythmia or the need for analgesia comprising a pharmaceutically acceptable carrier and an effective amount of a compound which is a suitable α₂-adrenoceptor modulator of the formula: 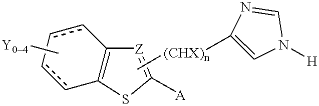
where
Z is N or C-Y (Y is absent where the imidazole containing substituent is attached at the 3-position);
n is 0 or 1;
Y is independently selected from the group consisting of hydrogen, C₁₋₄alkyl, bromine, chlorine, iodide, trifluoromethyl, C₁₋₄alkoxy, —SO₂NH₂ and nitro;
X is independently selected from the group consisting of hydrogen, hydroxy, C₁₋₄ alkyl, C₁₋₄ alkoxy, trifluoromethyl and phenyl; and
A is independently selected from the group consisting of hydrogen, C₁₋₄alkyl, chlorine, trifluoromethyl, C₁₋₄alkoxy and nitro, or is absent where the imidazole containing substituent is attached at the 2-position .

9. The pharmaceutical preparation of claim 8 wherein said human is suffering from the need for analgesia and said compound is a α₂-adrenoceptor agonist.

10. The pharmaceutical preparation of claim 8 wherein said human is suffering from depression and said compound is a α₂-adrenoceptor antagonist.
